(19) United States
(12) Patent Application Publication
(10) Pub. No.: US 2022/0403412 A1
(43) Pub. Date: Dec. 22, 2022

(54) Baculovirus Of Recombinant Superoxide Dismutase Gene, Preparation And Application Thereof

(71) Applicant: Hui Jing, Diamond Bar, CA (US)

(72) Inventor: Hui Jing, Diamond Bar, CA (US)

(21) Appl. No.: 17/346,086

(22) Filed: Jun. 11, 2021

(51) Int. Cl. C12N 15/86; C12N 5/07; C12N 9/02; C12N 9/10; C12N 15/52

(52) U.S. Cl. CPC C12N 15/86; C12N 5/0601; C12N 9/0089; C12Y 115/01001; C12N 9/1077; C12Y 204/02012; C12N 15/52; C12N 2510/02; C12N 2710/14043

(57) ABSTRACT

In one aspect, the present invention jointly expresses SOD and NAMPT proteins. The activity of SOD and NAMPT expressed by silkworm pupae inoculated with viruses is higher than that of silkworm pupae that are not inoculated with viruses, which proves that it is feasible to jointly express SOD and NAMPT with silkworms. It is expected to prepare an effective anti-aging drug.

### FIELD OF THE INVENTION

The present invention relates to the technology of producing polypeptide drugs by genetic engineering in the biotechnology pharmaceutical field.

### BACKGROUND OF THE INVENTION

Superoxide Dismutase (SOD), also known as orgotein, is an active substance derived from the living body, which can eliminate harmful substances produced by organisms in the metabolic process. Continuously supplementing SOD to the human body will have a special anti-aging effect. SOD was first isolated from bovine red blood cells in 1938, and so far, it has been studied for many years. In 1969, it was officially named SOD. As one of the most important enzymes in the human body, SOD's roles cannot be underestimated. Clinically, SOD can be used to treat and prevent acute inflammation, edema, oxygen poisoning (SOD can be pre-injected as preventive measures for workers who enter the hyperbaric oxygen chamber), oxygen poisoning treatment, autoimmune disease (early treatment), emphysema, irradiation sickness and radiation protection, senile cataract, etc., in addition, it has anti-aging functions. NAMPT (nicotinamide phosphoribosyltransferase), also known as visfatin, is widely found in adipose tissues, liver, spleen, kidneys, etc. It is a multifunctional protein that involves in the regulation of various physiological processes in the body and regulation of the NAD level of cardiomyocytes. Related experiments have shown that the NAD level of cardiomyocytes changes with the expression of NAMPT. NAMPT is an important substance in the process of NAD synthesis by cardiomyocytes. It can protect the heart from autophagy, prevent atherosclerosis and inhibit angiotensin II to induce myocardial hypertrophy. Studies on NAMPT are currently in its nascent state internationally, and if highly effective, healthy and safe NAMPT drugs are developed, they can be used in heart diseases, etc. We introduced the target gene of NAMPT and the target gene of heat-resistant SOD into the silkworm baculovirus, transfected silkworm pupa and its larva with the silkworm baculovirus, and expressed the two target genes jointly in the silkworms to generate a novel combination protein. It is a new approach to produce SOD and NAMPT.

### SUMMARY OF THE INVENTION

### Beneficial Effect

The virions prepared by the recombinant baculovirus provided by the present invention simultaneously express SOD and NAMPT proteins in silkworm larvae and silkworm pupae. The silkworm pupa powder prepared using the baculovirus can significantly increase the levels of SOD and GSH-Px activity in the blood of aged rats. Although the content of SOD and NAMPT proteins in the obtained silkworm pupa powder is limited, the effect is significant as following:
1. The present invention jointly expresses SOD and NAMPT proteins. The activity of SOD and NAMPT expressed by silkworm pupae inoculated with viruses is higher than that of silkworm pupae that are not inoculated with viruses, which proves that it is feasible to jointly express SOD and NAMPT with silkworms. It is expected to prepare an effective anti-aging drug.
2. The anti-aging drugs provided by the present invention have a low content of active ingredients, with low cost but remarkable effect. In the prior art, oral administration of 50,000 U/kg SOD has no significant difference in the SOD activity in the blood in rats, while the drug provided by the present invention has a significant effect when the SOD dosage is 8626 U/kg.
3. The anti-aging drug provided by the present invention is wrapped with silkworm pupa powder, has better oral effect; it needs not to add other excipients and the manufacturing process is simple.
4. After the recombinant baculovirus provided by the present invention infects silkworm larvae and pupae, the specific activity of SOD is significantly increased. Of which, the specific activity of SOD in hemolymph is increased by 14 times, which is much higher than the prior art (increased by 5 times).
5. The mechanism of action of SOD and NAMPT in vivo is not related. However, the silkworm pupa powder and drug test results provided in the present application show that the co-expression of the two proteins has significantly increased the antioxidant activity of SOD, with a significant anti-aging effect.

### DETAILED DESCRIPTION OF THE INVENTION

pET-28a-MnSOD plasmid (provided by the Silkworm Bioreactor Laboratory of Zhejiang Sci-Tech University), BmDH10Bac (provided by the Silkworm Bioreactor Laboratory of Zhejiang Sci-Tech University), pFastBac-Dual vector, Xho I, Kpn I, EcoR I, Hind III And BamH I, antibiotics, transfection reagent FuGene 6, serum. Unless otherwise specified, the reagents, vectors and strains required for the experiments could be commercially available.

### EXAMPLE 1

Construction of Recombinant Shuttle Vector pFastBac-Dual-MnSOD
I. Design of Primers for MnSOD Coding Region
   1. The heat-resistant MnSOD selected in the invention was an expression target gene. Its ORF sequence was as follows:

>MnSOD
ATGCCATTTGAATTGCCAGCATTGCCGTATCCGTATGATGCGCTTGAGC
CGCACATCGACAAAGAAACGATGAACATTCACCACACGAAGCACCATAA
CACATACGTTACAAATTTGAATGCGGCGCTTGAAGGGCATCCGGATTTG
CAAAACAAATCGCTCGAAGAATTGCTCAGCAATTTGGAAGCCCTTCCGG
AAAGCATTCGCACGGCGGTGCGCAACAACGGCGGCGGTCATGCAAACCA
CTCGCTTTTCTGGACGATTTTGTCGCCAAATGGCGGCGGTGAGCCGACG
GGTGAGCTGGCTGAGGCGATCAACAAAAAATTCGGCAGCTTCACCGCGT
TTAAAGACGAGTTTTCGAAAGCAGCGGCCGGCCGTTTCGGTTCTGGCTG
GGCATGGCTTGTCGTGAACAACGGCGAGCTGGAAATTACGAGCACGCCG
AACCAAGACTCGCCGATCATGGAAGGCAAAACGCCGATTCTCGGCTTGG
ACGTTTGGGAGCATGCGTACTACTTGAAATACCAAAACCGCCGTCCGGA
ATACATTGCCGCATTCTGGAACATTGTCAACTGGGACGAAGTGGCGAAA
CGGTACAGCGAAGCGAAAGCGAAGTAA

The primer was designed based on the ORF of the MnSOD gene, and the Xho I and Kpn I restriction sites were introduced respectively, as follows:

Upstream primer F (SEQ ID NO. 3):
CCTCGAGATGCCATTTGAATTGCCAG
(Xho I restriction site underlined)
Downstream primer R (SEQ ID NO. 4):
GGGTACCTTACTTCGCTTTCGCTTCGC
(Kpn I restriction site underlined)

### 2. The ORF Sequence of Target Gene NAMPT Expressed in the Invention was as Follows:

>NAMPT
ATGAATCCTGCGGCAGAAGCCGAGTTCAACATCCTCCTGGCCACCGACT
CCTACAAGGTTACTCACTATAAACAATATCCACCCAACACAAGCAAAGT
TTATTCCTACTTTGAATGCCGTGAAAAGAAGACAGAAAACTCCAAATTA
AGGAAGGTGAAATATGAGGAAACAGTATTTTATGGGTTGCAGTACATTC
TTAATAAGTACTTAAAAGGTAAAGTAGTAACCAAAGAGAAAATCCAGGA
AGCCAAAGATGTCTACAAAGAACATTTCCAAGATGATGTCTTTAATGAA
AAGGGATGGAACTACATTCTTGAGAAGTATGATGGGCATCTTCCAATAG
AAATAAAAGCTGTTCCTGAGGGCTTTGTCATTCCCAGAGGAAATGTTCT
CTTCACGGTGGAAAACACAGATCCAGAGTGTTACTGGCTTACAAATTGG
ATTGAGACTATTCTTGTTCAGTCCTGGTATCCAATCACAGTGGCCACAA
ATTCTAGAGAGCAGAAGAAAATATTGGCCAAATATTTGTTAGAAACTTC
TGGTAACTTAGATGGTCTGGAATACAAGTTACATGATTTTGGCTACAGA
GGAGTCTCTTCCCAAGAGACTGCTGGCATAGGAGCATCTGCTCACTTGG
TTAACTTCAAAGGAACAGATACAGTAGCAGGACTTGCTCTAATTAAAAA
ATATTATGGAACGAAAGATCCTGTTCCAGGCTATTCTGTTCCAGCAGCA
GAACACAGTACCATAACAGCTTGGGGGAAAGACCATGAAAAAGATGCTT
TTGAACATATTGTAACACAGTTTTCATCAGTGCCTGTATCTGTGGTCAG
CGATAGCTATGACATTTATAATGCGTGTGAGAAAATATGGGGTGAAGAT
CTAAGACATTTAATAGTATCAAGAAGTACACAGGCACCACTAATAATCA
GACCTGATTCTGGAAACCCTCTTGACACTGTGTTAAAGGTTTTGGAGAT
TTTAGGTAAGAAGTTTCCTGTTACTGAGAACTCAAAGGGTTACAAGTTG
CTGCCACCTTATCTTAGAGTTATTCAAGGGGATGGAGTAGATATTAATA
CCTTACAAGAGATTGTAGAAGGCATGAAACAAAAAATGTGGAGTATTGA
AAATATTGCCTTCGGTTCTGGTGGAGGTTTGCTACAGAAGTTGACAAGA
GATCTCTTGAATTGTTCCTTCAAGTGTAGCTATGTTGTAACTAATGGCC
TTGGGATTAACGTCTTCAAGGACCCAGTTGCTGATCCCAACAAAAGGTC
CAAAAAGGGCCGATTATCTTTACATAGGACGCCAGCAGGGAATTTTGTT
ACACTGGAGGAAGGAAAAGGAGACCTTGAGGAATATGGTCAGGATCTTC
TCCATACTGTCTTCAAGAATGGCAAGGTGACAAAAAGCTATTCATTTGA
TGAAATAAGAAAAAATGCACAGCTGAATATTGAACTGGAAGCAGCACAT
CATTAG

The primer was designed based on the ORF of the target gene NAMPT, and EcoRI and Hind III restriction sites were introduced respectively, as follow:

Upstream primer F (SEQ ID NO. 5):
GGAATTCATGAATCCTGCGGCAGAAG
(EcoRI site underlined)
Downstream primer R (SEQ ID NO. 6):
CCAAGCTTCTAATGATGTGCTGCTTCCAG
(Hind III site underlined)

3. Construction of the Recombinant Vector pFastBacDual-SOD-NAMPT

The ORF fragments (FIG. 1) of the heat-resistant SOD gene was obtained by amplifying the pET-28a-MnSOD plasmid (provided by the Bombyx mori Bioreactor Laboratory of Zhejiang Sci-Tech University) using PCR technology. The Xho I and Kpn I restriction sites were introduced at the ends of the fragments respectively, and the baculovirus shuttle vector pFastBacDual was ligated by double enzyme digestion to construct the recombinant vector pFastBacDual-SOD.

The ORF fragments of NAMPT enzyme target gene were obtained by amplifying human hepatocyte cDNA using PCR technology. The EcoR I and BamH I restriction sites were introduced at both ends of the fragment respectively, and the recombinant vector pFastBacDual-SOD-NAMPT was constructed by double restriction digestion and ligation. As shown from FIGS. 1 and 2, we successfully amplified the SOD and NAMPT target genes using PCR technology, and performed identification of the constructed recombinant vector pFastBacDual-SOD-NAMPT by double enzyme digestion and PCR, respectively, as shown in FIGS. 3, 4 and 5, indicating that we successfully constructed the recombinant vector pFastBacDual-SOD-NAMPT.

### 4. Construction and Identification of the Shuttle Vector BmBacmid-MnSOD-NAMPT of Recombinant Baculovirus

The recombinant vector pFastBacDual-SOD-NAMPT was transformed into BmDH10Bac E. coli competent cells containing baculovirus vector BmBacmid and transposable helper plasmid pMON7124 (provided by Zhejiang Sci-Tech University, refer to the patent CN201210037290.8 for details), and spread on a LB plate containing Kan , Gen, Tet, IPTG, and X-gal, cultured in a 37° C. incubator in the dark for 48 hours. The white colonies (FIG. 6) were picked and inoculated into LB liquid medium containing the above 3 antibiotics (namely Kan, Gen, Tet), and cultured while shaking at 37° C. , 220 rpm overnight, and then the recombinant BmBacmid DNA (BmBacmid-MnSOD-NAMPT) was extracted for cross- PCR verification, as shown in FIG. 7. Using the extracted recombinant vector as a template and different primer pairs (SEQ ID NO.3+SEQ ID NO.4 and SEQ ID NO.5+SEQ ID NO.6), the fragments that matched the expected results were obtained, indicating that the shuttle vector BmBacmid-MnSOD-NAMPT of the recombinant baculovirus was constructed successfully.

### EXAMPLE 2

Virions obtained by transfection of recombinant BmBacmind-SOD-NAMPT into silkworm cells and identification

The silkworm BmN cells were spread and cultured (FIG.8). The recombinant BmBacmind-SOD-NAMPT was transfected with Fugene 6 into the silkworm BmN cells. After 5 to 7 days, the cells floated and became large and round (FIG.9), indicating that cells developed disease and the recombinant baculovirus was obtained, then centrifuged to collect the supernatant, the virus solution. PCR identification was performed by adding the upstream and downstream primers F and R of MnSOD and NAMPT. As shown in FIG.10, the bands of the target gene ORF of SOD and NAMPT were amplified successfully, with the size consistent with the theoretical one, indicating that the recombinant virus vBmBacmind-MnSOD-NAMPT was successfully constructed.

### EXAMPLE 3

Preparation of Dual Recombinant Protein of SOD and NAMPT by Infecting Recombinant Baculovirus in vBmBacmind-MnSOD-NAMPT Silkworms

### 1. Virus Inoculation of Silkworm Larvae

The recombinant baculovirus solution obtained in Example 2 was dipped into the second joint of the tail of silkworm larvae with a syringe needle, and the silkworm state after the virus inoculation was recorded every day. One day after the silkworms were inoculated with the virus, their size became large and their food intakes increased. Over the time, the silkworms' food intakes decreased and their tails turned yellow, they became “irritable” and liked to crawl everywhere, indicating that the silkworm larvae were infected with viruses. Three days after the larvae became sick, the forefeet were cut to collect the hemolymph.

### 2. Virus Inoculation of Silkworm Pupae

The virus was inoculated at the second joint of the tail of silkworm pupae, and the status of the silkworm pupae after virus inoculation was recorded every day. After poisoning, there was no obvious change in the first two days. After the third day, the silkworm pupae gradually became soft and began to develop disease over the time. The silkworm pupae three days after sickness (generally 5 to 6 days after inoculation) were ground and homogenized, and centrifuged at 8000 rpm, and then the supernatant was lyophilized. The lyophilized powder was a silkworm pupa lyophilized powder containing SOD and NAMPT dual recombinant protein.

### EXAMPLE 4

### Detection of Specific Enzyme Activity of Dual Recombinant Proteins SOD and NAMPT Expressing in Silkworm Larvae

The forefeet of larvae were cut to collect hemolymph in three days after sickness. The total protein concentration in the silkworm hemolymph was detected by Beyotime Bradford protein concentration kits. According to the experimental method, a standard curve was drawn to obtain the equation: y=0.4697x+0.1219. The hemolymph to be detected was diluted by 500 times, and the protein absorption values of the four groups of silkworm hemolymph measured at the wavelength of 570 nm were as follows: the OD value of normal hemolymph 1 was 0.143, the OD value of normal hemolymph 2 was 0.142, the OD value of hemolymph 1 in grasserie silkworm was 0.261, and the OD value of hemolymph 2 in grasserie silkworm was 0.242, then the hemolymph protein concentrations of normal larvae and larvae with grasserie were calculated. After dilution by 500 times, the activity unit of SOD was determined using the total SOD activity test kit (WST-8 method). The absorbance measured at a wavelength of 450nm was shown in Table 1.

TABLE 1
SOD activity detection 450 nm wavelength absorbance value
Table 1 SOD activity test-the absorbance at a wavelength of 450 nm

| | Blank | Blank | | Blank | | Blank |
|---|---|---|---|---|---|---|
| | control | control | Sample | control | | control |
| No. | 1 | 2 | 1 | 3-1 | Sample2 | 3-2 |
| Absorbance | 0.826 | 0.093 | 0.625 | 0.193 | 0.408 | 0.128 |

According to the calculation methods of kit (a. Inhibition percentage=[(A _{Blank control 1}-A _{Blank control 2})-(A ₛₐₘₚₗₑ-A _{Blank control 3})]/(A _{Blank control 1}-A _{Blank control 2})×100%; b. SOD enzyme activity unit in the sample to be tested =inhibition percentage/(1-inhibition percentage) units), the hemolymph enzyme activity units of normal silkworm and the silkworm with grasserie were calculated, and results were 348.380 U/mL and 808.929 U/mL respectively, and the hemolymph specific enzyme activity of normal silkworm and silkworm with grasserie were 2.524 U/mg and 36.77U/mg respectively. Results showed that the SOD specific enzyme activity of the silkworms with grasserie was 14 times that of normal silkworms.

The specific enzyme activity of NAMPT in the silkworm hemolymph was detected using human NAMPT enzyme-linked immunoassay kit (Meimian), and the measured absorbance value of the reference standard was shown in Table 2, of which, the OD value of the blank hole was 0.055.

TABLE 2
Measured absorbance value of the reference standard

| No. | 1 | 2 | 3 | 4 | 5 |
|---|---|---|---|---|---|
| OD of reference | 0.115 | 0.229 | 0.424 | 0.811 | 2.022 |
| standard | | | | | |
| OD of reference | 0.06 | 0.174 | 0.369 | 0.756 | 1.967 |
| standard after | | | | | |
| zero setting | | | | | |
| Concentration of | 30 | 60 | 120 | 240 | 480 |
| reference standard | | | | | |
| (U/L) | | | | | |
| Concentration of | 0.625 | 1.25 | 2.5 | 5 | 10 |
| reference standard | | | | | |
| (ng/mL) | | | | | |

The linear relationship between the absorbance of the reference standard and the concentration (U/L) was plotted using the absorbance of the reference standard after zero setting, y =234.96x+29.704; and the linear relationship between the absorbance of the reference standard and the concentration (ng/mL): y=4.895x+0.6188. The silkworm hemolymph data were measured at a wavelength of 450 nm (as shown in Table 3), of which, the OD value of the blank hole was 0.057.

TABLE 3
Absorbance of silkworm hemolymph at a wavelength of 450 nm

| | Silkworm | Silkworm | Silkworm | Silkworm |
|---|---|---|---|---|
| | hemolymph | hemolymph | hemolymph | hemolymph |
| | sample 1 on | sample 2 on | sample 1 | sample 2 on |
| No. | Day 2 | Day 2 | on Day 4 | Day 4 |
| Sample OD value | 0.251 | 0.258 | 0.369 | 0.381 |
| OD value of | 0.194 | 0.201 | 0.312 | 0.324 |
| reference standard | | | | |
| after zero setting | | | | |
| NAMPT activity of | 75.28624 | 76.93096 | 103.01152 | 105.83104 |
| hemolymph (U/L) | | | | |
| NAMPT concen- | 1.56843 | 1.602695 | 2.14604 | 2.20478 |
| tration of hemo- | | | | |
| lymph (ng/mL) | | | | |

### EXAMPLE 5

### Detection of Enzyme Activity of Silkworm Pupa Freeze-Dried Powder Expressing Dual Recombinant Protein SOD and NAMPT

The silkworm pupae three days after the onset of disease (generally days after inoculation) were ground and homogenized and centrifuged at 8000 rpm, then the supernatant was freeze-dried; after dissolved and centrifuged, the total protein concentration and enzyme activity of the supernatant were determined using the same method, and then the specific enzyme activity was calculated. The total protein concentration of normal silkworm pupa sample supernatant was 50.500 mg/mL, the rotein concentration of diseased silkworm pupa sample was 26.500 mg/mL. The SOD activity was measured using the same method, and the enzyme activity unit of normal silkworm pupa was 0.677 U, the enzyme activity unit of diseased silkworm pupa was 2.286 U, then the specific enzyme activity of the normal silkworm pupa and diseased silkworm pupa was calculated, which was 6.703 U/mg and 43.132 U/mg respectively. The results showed that, the SOD specific enzyme activity of diseased silkworm pupae was 6.4 times that of normal silkworm pupae. The silkworm pupa as a bioreactor was more suitable for the expression of SOD. The specific enzyme activity of NAMPT protein in silkworm pupa was determined by the same method, results were shown in Table 4.

TABLE 4
Determination of specific enzyme activity
of NAMPT protein in silkworm pupa

| | | Powder | | |
|---|---|---|---|---|
| | Sample | sample | Sample | Sample |
| No. | 1 | 2 | 1 | 2 |
| Sample OD value | 0.251 | 0.274 | 0.321 | 0.352 |
| OD value of reference | 0.192 | 0.215 | 0.262 | 0.293 |
| standard after zero | | | | |
| setting | | | | |
| NAMPT enzyme activity of | 63.526 | 69.097 | 80.481 | 87.990 |
| lyophilized powder | | | | |
| dissolving solution(U/L) | | | | |
| NAMPT concentration of | 3.309 | 3.599 | 4.192 | 4.583 |
| lyophilized powder | | | | |
| dissolving solution (ng/mL) | | | | |
| NAMPT content in dry | 330.858 | 359.873 | 419.163 | 458.270 |
| powder (ng/g) | | | | |

From the above enzyme activity and concentration of NAMPT in silkworm pupa, it showed that the content of NAMPT expressed by the pupa of the diseased silkworms was higher than that pupa of the normal silkworms.

The vBmBacmid-MnSOD-NAMPT virus was inoculated in silkworm pupae in large quantities, and the MnSOD-NAMPT dual protein was expressed on a large scale. The virus was inoculated at the second joint of the e tail of the silkworm pupa by dipping with a needle, and the status of the silkworm pupae after virus inoculation was recorded every day. After inoculation, there was no obvious change in the first two days. After the third day, the silkworm pupae gradually became soft over the time. Among them, the blackened silkworm pupae were infected with bacteria, and there was no blackening if infected by viruses. The black silkworm pupae with bacterial infection should be removed in time. The specific enzyme activity of SOD and NAMPT proteins of silkworm pupa freeze-dried powder inoculated on a large scale was detected.

After large-scale inoculation of silkworm pupae with vBmBacmid-MnSOD-NAMPT virus, the juice was lyophilized into a dry powder, then 10 mg of silkworm pupa freeze-dried powder was dissolved in 1mL PBS solution, and the specific enzyme activity of recombinant SOD protein and NAMPT protein was determined by the same method as above, to detect the specificity of individual recombinant protein expression in different silkworm pupae. Results showed that after different batches of silkworm pupae were inoculated with the virus for the same time, the specific enzyme activity of expressed proteins was not much different.

### EXAMPLE 6

### Anti-Aging Experiment

Materials: Experimental animals: 36 old female rats aged 22 months, weighing 400-480g each; 12 female rats aged 4 months, weighing 200-250g each. Animals were purchased from the Animal Center of Zhejiang University of Traditional Chinese Medicine, and they were free to access to water and foods during the experiment. The laboratory temperature was (25±4)° C.
Drugs and reagents: The dual silkworm pupa powder expressing SOD and NAMPT (hereinafter referred to as dual silkworm pupa powder) obtained in example 3 and the commercially available common silkworm pupa powder, prepared with normal saline when using. SOD kits, glutathione peroxidase (GSH-Px) kits.
Methods: The elderly rats were randomly divided into the dual expression high-dose group, the dual expression low-dose group, and the elderly control group, 12 animals each. In addition, 4-month-elderly rats were used as the young control group. Rats were gavaged according to the body surface area of humans and animals, 200 and 500 mg/kg, once a day, for 4 weeks, rats in the control group was given common silkworm pupa powder. After the last gavage at the end of the 4th week, animals were fasted but were accessible to water. After anesthesia with 10% urethane, the blood was taken from the abdominal aorta. After standing for 4 hours at room temperature, the blood was centrifuged at 4000 rpm for 10 minutes to separate the serum for SOD and GSH-Px detection.
Statistical analysis of experimental data was performed using SPSS17.0 software, and ANOVA was performed for comparison between groups.
Regarding the effect on serum SOD and GSH-Px activities in the elderly rats, the serum SOD and GSH-Px activities in elderly rats were significantly decreased; after 4 weeks of intragastric treatment, compared with the elderly control group, the serum SOD and GSH-Px activities in the treatment group were significantly enhanced (P<0.05), as shown in Table 1.

TABLE 5
The effect of dual silkworm pupa powder containing recombinant SOD
and -NAMPT on the activities of serum SOD and GSH-Px in elderly rats

| Group | SOD activity (U/ml) | GSH-Px(U/ml) |
|---|---|---|
| Youth control group | 393.6 ± 46.57 | 216.57 ± 20.16 |
| Elderly control group | 234.8 ± 39.8 | 157.76 ± 18.91 |
| High-dose group | 357.8 ± 50.76 | 205.19 ± 23.89 |
| Low-dose group | 315.1 ± 36.99 | 189.54 ± 21.16 |

The virion prepared by the recombinant baculovirus provided by the present invention simultaneously expressed SOD and NAMPT proteins in silkworm larvae and silkworm pupae. The silkworm pupa powder prepared by using the baculovirus could significantly increase the levels of SOD and GSH-Px activities in the blood of the elderly rats. Although fewer effective ingredients needed to be given compared to the prior art, the anti-aging effect was indeed significantly improved.

Finally, it should be noted that only a few specific embodiments of the present invention are described above. Apparently, the present invention is not limited to the above embodiments, and many modifications and variations are possible. All modifications and variations that can be directly derived or associated by a person of ordinary skill in the art from the disclosure of the present invention should fall within the scope of protection of the present invention.

What is claimed is:

1. A recombinant baculovirus comprising sequences of a superoxide dismutase SOD gene and a nicotinamide phosphoribosyltransferase NAMPT gene, and the SOD gene is a thermophile SOD gene with a sequence as shown in SEQ ID NO.1; the NAMPT gene sequence is shown in SEQ ID NO.2.

2. The recombinant baculovirus of claim 1, wherein the virus is a Bombyx mori baculovirus.

3. The recombinant baculovirus of claim 2, wherein the virus is obtained by the following steps:
designing a primer according to SEQ ID NO.1 and SEQ ID NO.2, ligating the ORF of SOD and NAMPT to the same baculovirus vector using double enzyme digestion and molecular cloning methods, and constructing a recombinant vector.

4. The recombinant baculovirus of claim 3, wherein the primer is as shown in SEQ ID NO.3, SEQ ID NO.4, SEQ ID NO.5, and SEQ ID NO.6.

5. The recombinant baculovirus of claim 3, wherein the baculovirus vector is pFastBacDual.

6. The recombinant baculovirus of claim 1, wherein a recombinant protein is obtained by the recombinant baculovirus infecting silkworm pupa or silkworm or silkworm cells.

7. The recombinant baculovirus of claim 6, wherein the silkworm pupa powder is prepared by the following steps:
(1) infecting silkworm cells with the recombinant baculovirus to obtain virions;
(2) Inoculating silkworm pupae with the virions of step (1), squeezing juice after 5 to 6 days, centrifuging at 8000 rpm to obtain a supernatant, and freeze-drying the supernatant, to obtain a freeze-dried powder of silkworm pupa.


SEQUENCE LISTING (4 sequences)

SEQ ID NO: 1 - DNA, 615 bp, Artificial Sequence
  MnSOD
atgccatttgaattgccagcattgccgtatccgtatgatgcgcttgagccgcacatcgacaaagaaacgatgaacattcaccacacgaagcaccataacacatacgttacaaatttgaatgcggcgcttgaagggcatccggatttgcaaaacaaatcgctcgaagaattgctcagcaatttggaagcccttccggaaagcattcgcacggcggtgcgcaacaacggcggcggtcatgcaaaccactcgcttttctggacgattttgtcgccaaatggcggcggtgagccgacgggtgagctggctgaggcgatcaacaaaaaattcggcagcttcaccgcgtttaaagacgagttttcgaaagcagcggccggccgtttcggttctggctgggcatggcttgtcgtgaacaacggcgagctggaaattacgagcacgccgaaccaagactcgccgatcatggaaggcaaaacgccgattctcggcttggacgtttgggagcatgcgtactacttgaaataccaaaaccgccgtccggaatacattgccgcattctggaacattgtcaactgggacgaagtggcgaaacggtacagcgaagcgaaagcgaagtaa

SEQ ID NO: 2 - DNA, 1476 bp, Artificial Sequence
  NAMPT
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcaagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattag

SEQ ID NO: 3 - DNA, 26 bp, Artificial Sequence
  Upstream primer F
ggaattcatgaatcctgcggcagaag

SEQ ID NO: 4 - DNA, 29 bp, Artificial Sequence
  Downstream primer R
ccaagcttctaatgatgtgctgcttccag